(19) United States
(12) Patent Application Publication
(10) Pub. No.: US 2011/0208117 A1
(43) Pub. Date: Aug. 25, 2011

(54) Catheter

(76) Inventor: Charles R. Hawkins, (US)

(21) Appl. No.: 12/712,375

(22) Filed: Feb. 25, 2010

(51) Int. Cl. A61M 25/14; H04Q 5/22

(52) U.S. Cl. 604/28; 604/500; 604/523; 604/27; 340/10.42

(57) ABSTRACT

A catheter is disclosed. The catheter has a body having a proximal end and a distal end. The body encloses a plurality of independent lumens which extend longitudinally from the proximal end to the distal end. One of the independent lumens is sealed at the distal end which allows for the insertion of an agent. A tag used to locate the distal end of a mono or multiple lumen catheter is also disclosed. A tip used in conjunction with a mono or multiple lumen catheter is also disclosed. Methods of use are also disclosed.

### RELATED APPLICATIONS

NOT APPLICABLE

### STATEMENT REGARDING FEDERALLY SPONSORED RESEARCH OR DEVELOPMENT

NOT APPLICABLE

### REFERENCE A MICROFICHE APPENDIX

NOT APPLICABLE

### FIELD OF THE INVENTION

The invention relates to catheters used in medicine for the delivery and/or withdrawal of one or more substances.

### BACKGROUND

Catheters are frequently used in medicine for the delivery and/or withdrawal of a variety of substances for medical diagnosis and/or medical treatment. Catheters are used for the delivery and/or withdrawal of one or more substances including fluids, blood and gases such as air from anywhere in the body. Soft bodied catheters often used to accomplish the delivery and/or withdrawal of substances to a patient offer several benefits including: increased safety, reduced tissue trauma and greater comfort. However, the softer and more flexible the catheter, the greater the movement of the catheter within the body. Such movement can irritate and injure as well as cause unintentional occlusion of the catheter's lumen(s) by contact with bodily tissues, vessel walls, and chambers of the heart among other internal anatomy. In addition, the suction used to withdraw a variety of substances can irritate and injure bodily tissues being pulled into contact with the catheter lumens.

A peripherally inserted central catheter (PICC), one example of a venous catheter, is a soft bodied open lumen catheter. A PICC is typically inserted via a peripheral vein of an upper extremity with the distal end of the PICC residing in the central venous system, generally the superior vena cava. Recently there has been an increased demand for power injection (PI) via the PICC and other venous catheters. PI is for the rapid infusion of a contrast media for enhancing images obtained by various radiographic or other means. Such contrast media is a fluid often having a higher viscosity than standard intravenous fluids. The rapid infusion of contrast is necessary to ensure the contrast is in the proper location long enough to allow for the imaging process and not carried away by blood or other bodily fluids prior to the acquisition of the images. With the rapid infusion of a more viscous fluid via a soft bodied catheter, the distal end of the catheter tends to move in an erratic pattern much like a fire hose with a full stream of water and no firemen to control it. Permanently stiffening the soft bodied flexible catheter to reduce the firehosing effect increases the likelihood of the catheter causing irritation and damage to the blood vessels upon contact. The firehosing effect often causes catheter tip malposition with potentially dangerous results depending on the degree of malposition, the type of intravenous infusion and the state of health of the patient and his or her blood vessels.

A catheter is needed that allows temporary changes to the stiffness of the catheter to accommodate power injection by the addition and removal of an agent. Additionally, a catheter is needed that reduces tissue irritation and injury while keeping the lumen or lumens of such catheter open for its desired use. Also, a catheter is needed that allows instant and repeated identification of the position of the distal end of a catheter to confirm placement of such catheter while inside a patient. A catheter is also needed that has embedded technology which allows the end of the catheter to be located and which also allows for information storage that may be communicated to medical personnel when needed.

### BRIEF SUMMARY

The present invention discloses a catheter having a body. The body has a proximal end and a distal end. The body encloses a plurality of independent lumens which extend longitudinally from the proximal end to the distal end. One of the independent lumens is sealed at the distal end which allows for the insertion of an agent. In one embodiment, the catheter has two independent lumens. In another embodiment, the catheter has three independent lumens.

The agent may be a solid, liquid or a gas. In one embodiment, the agent is a solid and may be a wire. The agent in solid form has a distal end.

In one embodiment, a tag may be attached to the distal end of the agent in solid form. In one embodiment, the tag may be magnetic. In another embodiment, the tag may be non-magnetic. In one embodiment, the tag is a radio frequency identification tag.

In one embodiment, a tag may be embedded in the body of the catheter at or near the distal end of the body. In one embodiment, the tag may be magnetic. In another embodiment, the tag may be non-magnetic. In one embodiment, the tag is a radio frequency identification tag.

The catheter may have a tip contiguous to the distal end of the body. In one embodiment, a tag is embedded in the tip. The tag may be magnetic or non-magnetic. In one embodiment, the tag is a radio frequency identification tag.

In another embodiment, a catheter for use in the delivery and/or the withdrawal of a variety of substances to a patient is also disclosed. The catheter has a body. The body has a proximal end and a distal end. The body has at least one lumen which extends longitudinally from the proximal end to the distal end. A tip is contiguous to the distal end of the body. In one embodiment, the proximal end of the body of a catheter is attached to a totally implantable venous access system.

A tag may be embedded in the tip. In another embodiment, a tag may be embedded in the body of the catheter at or near the distal end of the body. The tag may be magnetic or non-magnetic. In one embodiment, the tag is a radio frequency identification tag. In one embodiment, the proximal end of the body of a catheter is attached to a totally implantable venous access system.

A method for stiffening a catheter is disclosed. The method comprises the following steps: (a) obtaining a catheter for use in the delivery and/or the withdrawal of a variety of substances to a patient, wherein the catheter comprises a body having a proximal end and a distal end wherein the body encloses a plurality of independent lumens which extend longitudinally from the proximal end to the distal end, wherein at least one of the lumens is sealed at the distal end which allows for the insertion of an agent; (b) inserting the agent into the at least one of the lumens that is sealed at the distal end; (c) inserting the catheter into a patient; (d) securing the catheter to the patient; (e) delivering and/or withdrawing a substance to and/or from the patient; and (f) removing the agent. The agent may be a solid, liquid or gas.

In one embodiment, the method further comprises the steps: (g) reinserting the agent into the at least one of the lumens that is sealed at the distal end; (h) delivering and/or withdrawing a substance to and/or from a patient; and (i) removing the agent. The agent may be a solid, liquid or gas.

A method is also disclosed for stiffening a catheter for use in the delivery and/or the withdrawal of a variety of substances to a patient, wherein the catheter comprises a body having a proximal end and a distal end wherein the body encloses a plurality of independent lumens which extend longitudinally from the proximal end to the distal end, wherein at least one of the lumens is sealed at the distal end which allows for the insertion of a agent, wherein the catheter has previously been inserted and secured to a patient. The method comprises the following steps: (a) inserting the agent into the at least one of the lumens that is sealed at the distal end; (b) delivering and/or withdrawing a substance to and/or from a patient; and (c) removing the agent. The agent may be a solid, liquid or gas.

A method for identifying the position of and/or communicating with the distal end of a catheter is also disclosed. The method comprises the following steps: (a) obtaining a catheter for use in the delivery and/or the withdrawal of one or more substances to a patient, wherein the catheter has a body having a proximal end and a distal end wherein the body encloses at least one lumen which extends longitudinally from the proximal end to the distal end, wherein a tag is located near or at the distal end of the body; (b) inserting the catheter into a patient; (c) securing the catheter to the patient; and (d) using identification technology to identify the position of and/or communicate with the tag located near or at the distal end of the catheter. In one embodiment, the tag is embedded in a tip which is contiguous to the distal end of the body.

A method for identifying the position of and/or communicating with a tag located on an agent which is located at or near the distal end of a catheter is also disclosed. The method comprises the following steps: (a) obtaining a catheter for use in the delivery and/or the withdrawal of a variety of substances to a patient, wherein the catheter has a body having a proximal end and a distal end wherein the body encloses a plurality of lumens which extend longitudinally from the proximal end to the distal end, wherein at least one of the lumens is sealed at the distal end which allows for the insertion of an agent, wherein the agent has a proximal end and a distal end, wherein a tag is attached to the distal end of the agent; (b) inserting the agent into one of the lumens which is sealed at the distal end; (c) inserting the catheter into a patient; (d) securing the catheter to the patient; (e) using identification technology to identify the position of and/or communicate with the tag; and (f) removing the agent. The agent may be a solid.

In one embodiment, the method further comprises the steps: (g) reinserting the agent into one of the lumens which is sealed at the distal end; and (h) using identification technology to identify the position of and/or communicate with the tag. The agent may be a solid.

A method for identifying the position of and/or communicating with a tag located on an agent located at or near the distal end of a catheter is disclosed wherein the catheter is for use in the delivery and/or the withdrawal of a variety of substances to a patient, wherein the catheter has a body having a proximal end and a distal end wherein the body encloses a plurality of lumens which extend longitudinally from the proximal end to the distal end, wherein at least one of the lumens is sealed at the distal end which allows for the insertion of an agent, wherein the agent has a distal end, wherein a tag is attached to the distal end of the agent, wherein the catheter has been inserted into the patient. The method comprises the following steps: (a) inserting the agent into one of the lumens which is sealed at the distal end and (b) using identification technology to identify the position of and/or communicate with the tag. The agent may be a solid.

A method for preventing the distal end of a catheter from irritating or injuring any body duct, cavity or tissue, and/or causing open lumen occlusion, the method comprising: (a) obtaining a catheter for the delivery and/or withdrawal of one or more substances to a patient, wherein the catheter comprises a body having a proximal end and a distal end wherein the body encloses at least one independent lumen which extends longitudinally from the proximal end to the distal end, wherein a tip is contiguous to the distal end of the body; (b) inserting the catheter into a patient; (c) securing the catheter to the patient; and (d) delivering and/or withdrawing a substance to and/or from the patient.

### BRIEF DESCRIPTION

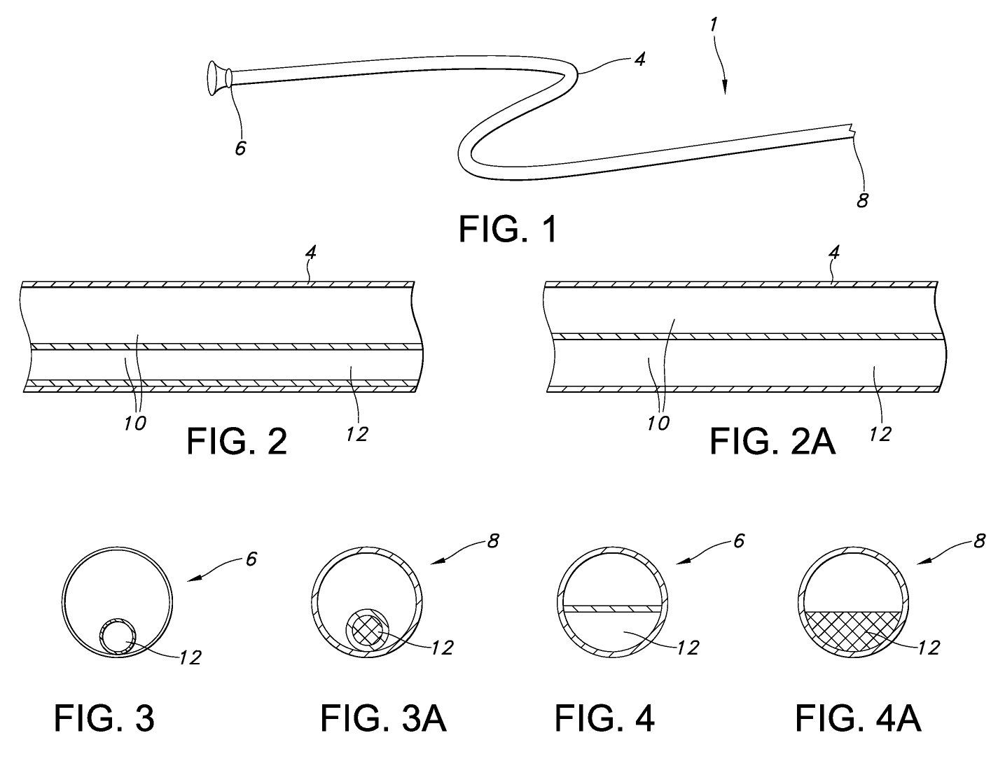

FIG. 1 is a side perspective view of a catheter;

FIG. 2 is an enlarged longitudinal cross-sectional view of the midsection of the catheter seen in FIG. 1 in accordance with a first embodiment;

FIG. 2A is an enlarged longitudinal cross-sectional view of the midsection of the catheter seen in FIG. 1 in accordance with a second embodiment;

FIG. 3 is an enlarged sectional view of the proximal end of the catheter seen in FIG. 1 in accordance with a first embodiment;

FIG. 3A is an enlarged sectional view of the distal end of the catheter seen in FIG. 1 in accordance with a first embodiment;

FIG. 4 is an enlarged sectional view of the proximal end of the catheter seen in FIG. 1 in accordance with a second embodiment;

FIG. 4A is an enlarged sectional view of the distal end of the catheter seen in FIG. 1 in accordance with a second embodiment;

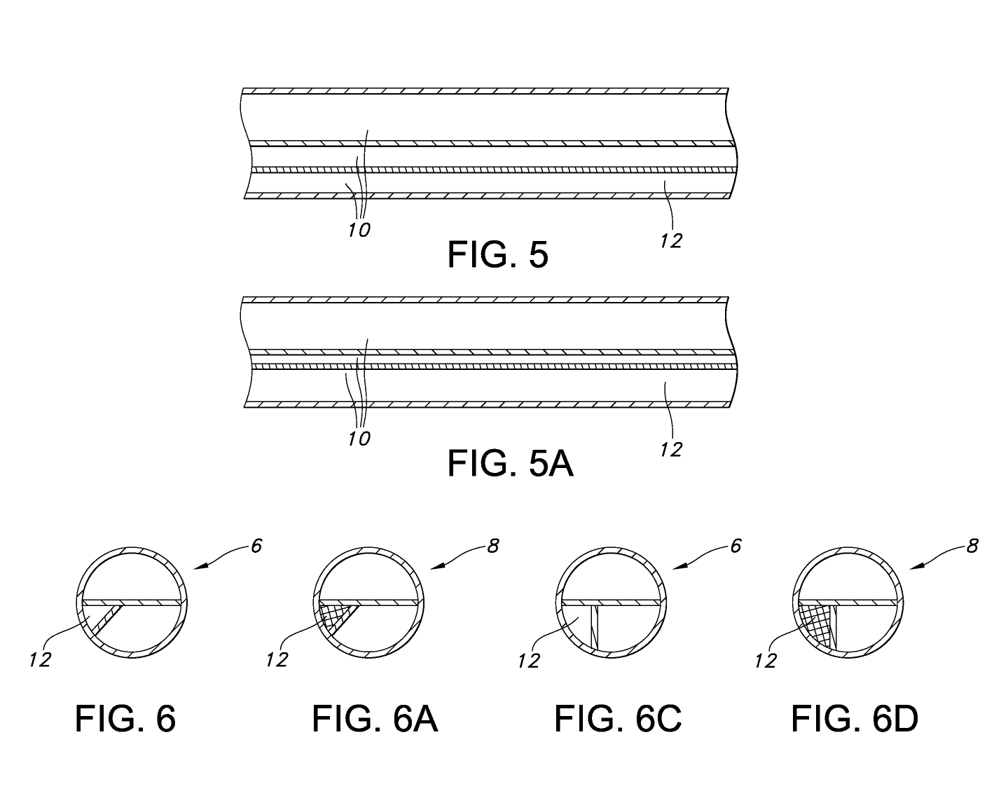

FIG. 5 is an enlarged longitudinal cross-sectional view of the midsection of a catheter in accordance with a third embodiment;

FIG. 5A is an enlarged longitudinal cross-sectional view of the midsection of a catheter in accordance with a fourth embodiment;

FIG. 6 is an enlarged sectional view of the proximal end of the catheter seen in FIG. 5;

FIG. 6A is an enlarged sectional view of the distal end of the catheter seen in FIG. 5;

FIG. 6C is an enlarged sectional view of the proximal end of the catheter seen in FIG. 5A;

FIG. 6D is an enlarged sectional view of the distal end of the catheter seen in FIG. 5A;

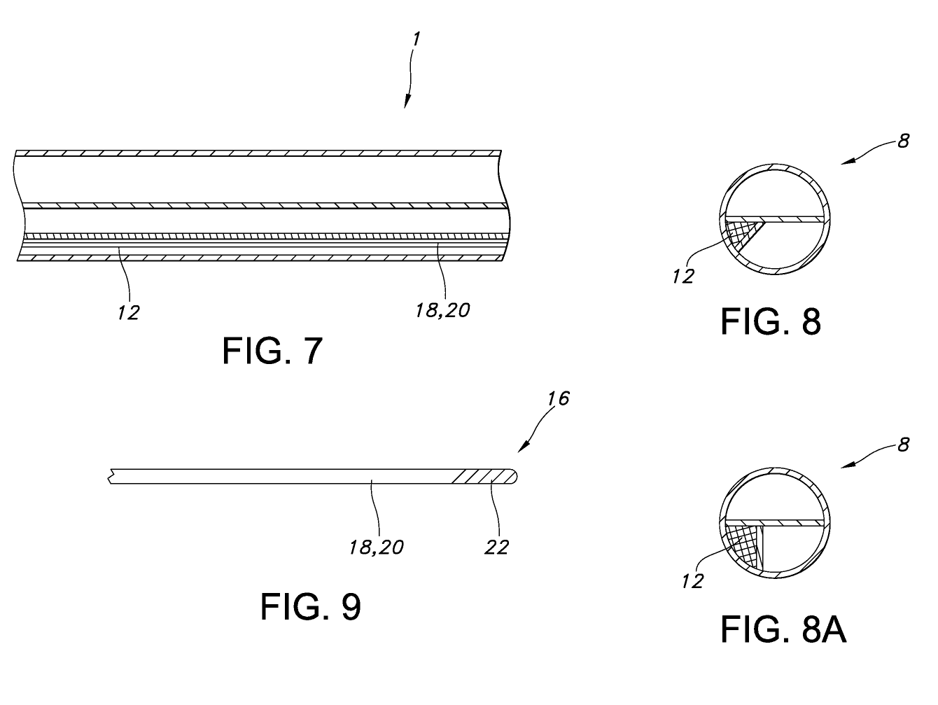

FIG. 7 is an enlarged longitudinal cross-sectional view of an agent inserted into a catheter;

FIG. 8 is an enlarged sectional view of the distal end of the catheter seen in FIG. 7 in accordance with a first embodiment;

FIG. 8A is an enlarged sectional view of the distal end of the catheter seen in FIG. 7 in accordance with a second embodiment;

FIG. 9 is a perspective view of an agent with a tag;

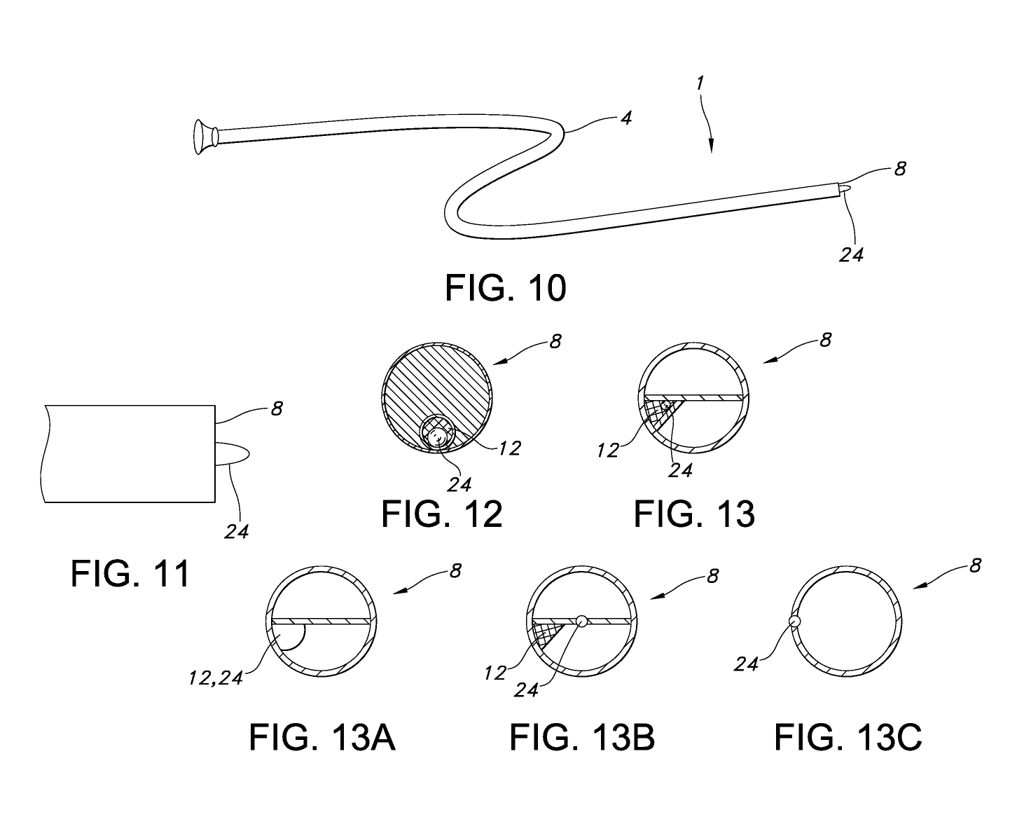

FIG. 10 is a perspective view of a catheter with a tip contiguous to the distal end of the body;

FIG. 11 is an enlarged perspective side view of a tip as seen in FIG. 10;

FIG. 12 is an enlarged sectional view of a distal end of a catheter depicting a tip contiguous to the distal end of a body;

FIG. 13 is an enlarged sectional view of a distal end of a catheter depicting a tip contiguous to the distal end of a body in accordance with a second embodiment;

FIG. 13A is an enlarged sectional view of a distal end of a catheter depicting a tip contiguous to the distal end of a body in accordance with a third embodiment;

FIG. 13B is an enlarged sectional view of a distal end of a catheter depicting a tip contiguous to the distal end of a body in accordance with a fourth embodiment;

FIG. 13C is an enlarged sectional view of a distal end of a catheter depicting a tip contiguous to the distal end of a body;

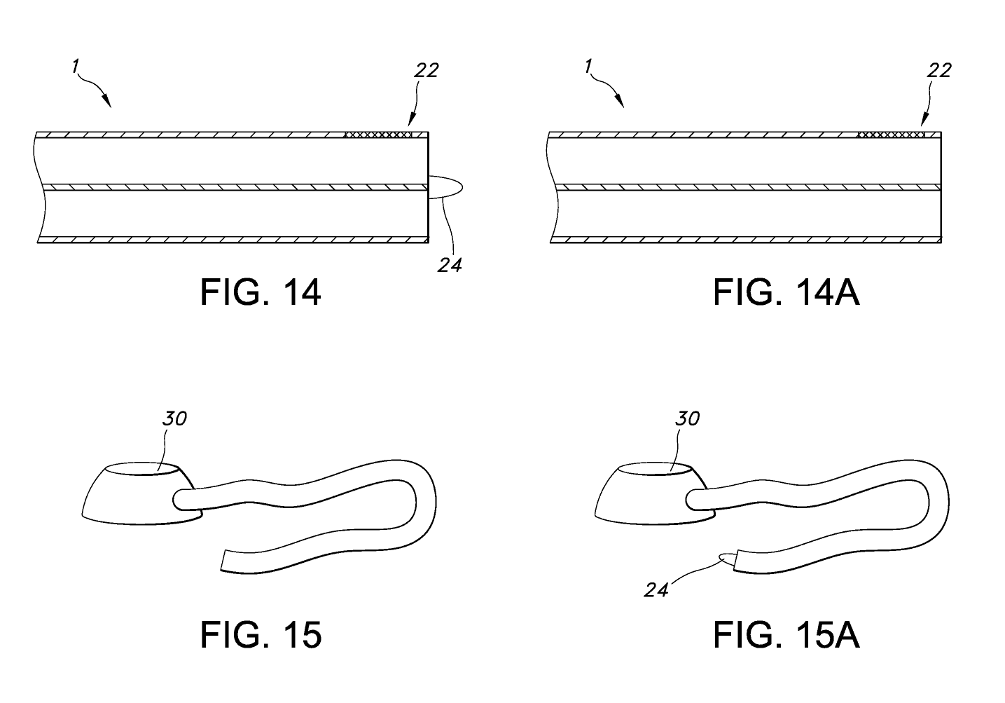

FIG. 14 is a perspective view of a tag embedded in the catheter at or near the distal end of the catheter;

FIG. 14A is a perspective view of a tag embedded in the catheter at or near the distal end of the catheter in accordance with a second embodiment;

FIG. 15 is a side perspective view of a catheter attached to a totally implantable venous access system;

FIG. 15A is a side perspective view of a catheter attached to a totally implantable venous access system in accordance with a second embodiment; and

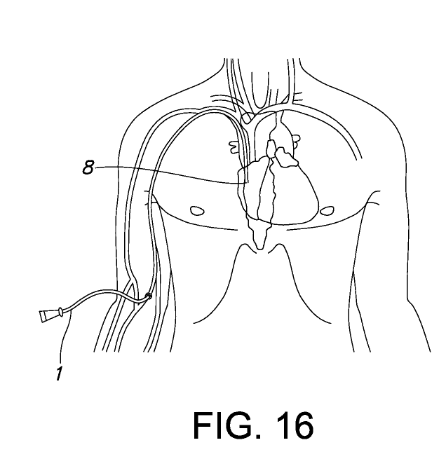

FIG. 16 is a perspective view of a catheter inserted via a peripheral vein of an upper extremity with the distal end of a catheter residing in the central venous system.

### DETAILED DESCRIPTION

Referring to FIG. 1, catheter 1 allows for the modification of the structural strength or stiffness of catheter 1. Catheter 1 has a flexible body 4. Body 4 is preferably made of a polymer such as polyurethane, but any other material such as silicone may be used as desired by one of skill in the art. Body 4 has proximal end 6 and distal end 8. Catheter 1 is secured to a patient's body at proximal end 6 by a suture or adhesive pad but any other form of securing catheter 1 to a patient's body may be used as desired by one of skill in the art. Referring now to FIGS. 2 to 2A and 5 to 5A, body 4 encloses a plurality of independent lumens 10 which extend longitudinally from proximal end 6 to distal end 8. Such plurality of independent lumens 10 allow for the delivery and/or the withdrawal of a variety of substances to a patient. In one embodiment, catheter 1 has two independent lumens 10 seen in FIGS. 2 and 2A. In another embodiment, catheter 1 has three independent lumens 10 seen in FIGS. 5 and 5A. Catheter 1 may have any number of independent lumens 10, such as four or five, as desired by one of skill in the art depending on the need and use for catheter 1.

Referring now to FIGS. 2-6D, one 12 of the plurality of independent lumens 10 is sealed at distal end 8. The seal at distal end 8 (see FIGS. 3A, 4A, 6A and 6D for multiple embodiments) allow temporary changes to the stiffness of catheter 1 by the addition and removal of agent 18. One 12 of independent lumens 10 may be sealed in any manner as desired by one of skill in the art as long as such seal prevents agent 18 from exiting the distal end 8 of catheter 1 and prevents any substance from entering or exiting the body of a patient through sealed lumen 12. The size of sealed lumen 12 may vary as desired by one skilled in the art.

In one embodiment, proximal end 6 is open (see FIGS. 3A, 4A, 6A and 6D for multiple embodiments). In one embodiment, proximal end 6 has a detachable valve (not shown). In another embodiment proximal end 6 has an attached valve (not shown).

Referring now to FIGS. 7 to 9, agent 18 may be inserted into one 12 of independent lumens 10 to change the structural strength or stiffness of catheter 1. One 12 of independent lumens 10 will be marked as desired by one of skill in the art to indicate such lumen 12 is sealed at distal end 8. Agent 18 is inserted into proximal end 6 of one 12 of independent lumens 10. Distal end 8 of one 12 of independent lumens 10 is sealed (see FIGS. 8 to 8A) which prevents agent 18 from exiting distal end 8 of catheter 1 and contacting and injuring and/or contaminating the patient's body anatomy and/or body fluids. Agent 18 may be a flexible solid such as a wire 20 (see FIGS. 7 and 9). Agent 18 may be any other flexible solid, such as a plastic, as desired by one of skill in the art. In another embodiment, agent 18 may be a liquid such as water. In still another embodiment, agent 18 may be a gas such as carbon dioxide.

In one embodiment, tag 22 is attached at the distal end 16 of stiffening agent 18 (such as wire 20 shown in FIG. 9) that will be inserted via the proximal end 6 of one 12 of independent lumens 10 and extend in to the distal end 8 of one 12 of independent lumens 10. Tag 22 may be any identification mechanism that can be located by identification technology, such as sensors. Tag 22 in conjunction with identification technology may be used to locate the position of the distal end 8 of catheter 1. Tag 22 may also be used for information storage, including personal identification, medical history, medications, allergies and contact information. In one embodiment, tag 22 is magnetic. In one embodiment, tag 22 is non-magnetic such as a radio frequency identification (RFID) tag.

Referring to FIGS. 10 to 13C, in one embodiment, tip 24 is contiguous to distal end 8 of catheter 1. Tip 24 prevents distal end 8 from making certain contact with body duct, cavity or tissues that would result in irritation and/or injury to such body duct, cavity or tissues and also from making certain contact that would occlude the open lumen or lumens 10. In one embodiment, tip 24 is made of a polymer such as polyurethane, but any other material such as silicone may be used as desired by one of skill in the art. In one embodiment, catheter 1 has one tip 24 but any number of tips may be used as desired by one of skill in the art to prevent irritation and injury to body anatomy by distal end 8 and/or to prevent open lumen occlusion. Tip 24 may be located anywhere on distal end 8 of catheter 1 as long as tip 24 prevents distal end 8 of catheter 1 from injuring and/or irritating any body duct, cavity or tissue and/or from causing open lumen occlusion. The size of tip 24 is dependent on the size of distal end 8 of catheter 1. The size of tip 24 may be any size as long as tip 24 prevents distal end 8 from injuring and/or irritating any body duct, cavity or tissues and/or from causing open lumen occlusion. Referring to FIGS. 12 to 13, catheter 1 is multi luminal and tip 24 is located on distal end 8 and covers a portion of sealed lumen 12. Referring to FIG. 13A, catheter 1 is multi luminal and tip 24 is located on distal end 8 and covers entire sealed lumen 12. Referring now to FIG. 13B, catheter 1 is multi luminal and tip 24 is located on body 4 of distal end 8 of catheter 1. Referring to FIG. 13C, in one embodiment, catheter 1 is monoluminal and tip 24 is located on body 4 of distal end 8 of catheter 1.

In one embodiment, tag 22 is embedded in tip 24. In another embodiment, tag 22 may be embedded in the body 4 at or near the distal end 8 of body 4 (see FIGS. 14 and 14A). Tag 22 is used for information storage, including personal identification, medical history, medications, allergies and contact information. Tag 22 may be any identification mechanism that can be located by identification technology, such as sensors. Tag 22 in conjunction with identification technology may be used to locate the position of the distal end 8 of catheter 1. In one embodiment, tag 22 may be magnetic. In one embodiment, tag 22 may be non-magnetic such as a radio frequency identification (RFID) tag.

In one embodiment, proximal end 6 of catheter 1 is attached to a totally implantable venous access system 30 (see FIGS. 15 and 15A).

A method for stiffening a catheter is disclosed. The method comprises the following steps: (a) obtaining catheter 1 for use in the delivery and/or the withdrawal of a variety of substances to a patient, wherein catheter 1 comprises body 4 having proximal end 6 and distal end 8 wherein body 4 encloses a plurality of independent lumens 10 which extend longitudinally from proximal end 6 to distal end 8, wherein at least one 12 of lumens 10 is sealed at distal end 8 which allows for the insertion of agent 18; (b) inserting agent 18 into at least one 12 of lumens 10 that is sealed at distal end 8; (c) inserting catheter 1 into a patient; (d) securing catheter 1 to the patient; (e) delivering and/or withdrawing a substance to and/or from the patient; and (f) removing agent 18. Agent 18 may be a solid, liquid or gas.

In one embodiment, the method further comprises the steps: (g) reinserting agent 18 into the at least one 12 of lumens 10 that is sealed at distal end 8; (h) delivering and/or withdrawing a substance to and/or from the patient; and (i) removing the agent 18. Agent 18 may be a solid, liquid or gas.

A method is also disclosed for stiffening catheter 1 for use in the delivery and/or the withdrawal of a variety of substances to a patient, wherein catheter 1 comprises body 4 having proximal end 6 and distal end 8 wherein body 4 encloses a plurality of independent lumens 10 which extend longitudinally from proximal end 6 to distal end 8, wherein at least one 12 of lumens 10 is sealed at distal end 8 which allows for the insertion of agent 18, wherein catheter 1 has previously been inserted and secured to a patient. The method comprises the following steps: (a) inserting agent 18 into the at least one 12 of lumens 10 that is sealed at distal end 8; (b) delivering and/or withdrawing a substance to and/or from a patient; and (c) removing agent 18. Agent may be a solid, liquid or gas.

A method for identifying the position of and/or communicating with distal end 8 of catheter 1 is also disclosed. The method comprises the following steps: (a) obtaining catheter 1 for use in the delivery and/or the withdrawal of one or more substances to a patient, wherein catheter 1 has body 4 having proximal end 6 and distal end 8 wherein body 4 encloses at least one independent lumen 10 which extends longitudinally from proximal end 6 to distal end 8, wherein tag 22 is located near or at distal end 8 of body 4; (b) inserting catheter 1 into a patient; (c) securing catheter 1 to the patient; and (d) using identification technology to identify the position of and/or communicate with tag 22 located near or at distal end 8 of catheter 1. In one embodiment, tag 22 is embedded in tip 24 which is contiguous to distal end 8 of body 4.

A method for identifying the position of and/or communicating with tag 22 located on agent 18 is also disclosed. The method comprises the following steps: (a) obtaining catheter 1 for use in the delivery and/or the withdrawal of a variety of substances to a patient, wherein catheter 1 has body 4 having proximal end 6 and distal end 8 wherein body 4 encloses a plurality of lumens 10 which extend longitudinally from proximal end 6 to distal end 8, wherein at least one 12 of lumens 10 is sealed at distal end 8 which allows for the insertion of agent 18, wherein agent 18 has proximal end 14 and distal end 16, wherein tag 22 is attached to distal end 16 of agent 18; (b) inserting agent 18 into one 12 of lumens 10 which is sealed at distal end 8; (c) inserting catheter 1 into the patient; (d) securing catheter 1 to the patient; (e) using identification technology to identify the position of and/or communicate with distal end 8 of catheter 1; and (f) removing agent 18. In one embodiment, agent 18 is a solid.

In one embodiment, the method further comprises the steps: (g) reinserting agent 18 into one 12 of lumens 10 which is sealed at distal end 8; and (h) using identification technology to identify the position of and/or communicate with tag 22. In one embodiment, agent 18 is a solid.

A method for identifying the position of and/or communicating with tag 22 located on agent 18 which is at or near distal end 8 of catheter 1 is disclosed wherein catheter 1 is for use in the delivery and/or the withdrawal of a variety of substances to a patient, wherein catheter 1 has body 4 having proximal end 6 and distal end 8 wherein body 4 encloses a plurality of lumens 10 which extend longitudinally from proximal end 6 to distal end 8, wherein at least one 12 of lumens 10 is sealed at distal end 8 which allows for the insertion of agent 18, wherein agent 18 has distal end 16, wherein tag 22 is attached to distal end 16 of agent 18, wherein catheter 1 has been inserted into the patient. The method comprises the following steps: (a) inserting agent 18 into one 12 of the lumens which is sealed at distal end 8 until distal end 8 is at or near distal end 8 of catheter 1 and (b) using identification technology to identify the position of and/or communicate with tag 22. In one embodiment, agent 18 is a solid.

A method for preventing distal end 8 of catheter 1 from irritating or injuring any body duct, cavity or tissue, and/or causing open lumen occlusion, the method comprising: (a) obtaining catheter 1 for the delivery and/or withdrawal of one or more substances to a patient, wherein catheter 1 comprises body 4 having proximal end 6 and distal end 8 wherein body 4 encloses at least one independent lumen 10 which extends longitudinally from proximal end 6 to distal end 8, wherein tip 24 is contiguous to distal end 8 of body 4; (b) inserting catheter 1 into a patient; (c) securing catheter 1 to the patient; and (d) delivering and/or withdrawing a substance to and/or from the patient.

The foregoing disclosure has been set forth merely to illustrate the invention and is not intended to be limiting. Since modifications of the disclosed embodiments incorporating the spirit and substance of the invention may occur to persons skilled in the art, the invention should be construed to include everything within the scope of the disclosed invention and equivalents thereof.

We claim:

1. A catheter for use in the delivery and/or the withdrawal of one or more substances to a patient, wherein the catheter comprises a body having a proximal end and a distal end wherein the body encloses a plurality of independent lumens which extend longitudinally from the proximal end to the distal end, wherein at least one of the lumens is sealed at the distal end which allows for the insertion of an agent.

2. The catheter of claim 1 wherein the catheter has two independent lumens.

3. The catheter of claim 1 wherein the catheter has three independent lumens.

4. The catheter of claim 1 wherein the agent is a solid and has a distal end.

5. The catheter of claim 4 wherein the agent is a wire.

6. The catheter of claim 1 wherein the agent is a liquid.

7. The catheter of claim 1 wherein the agent is a gas.

8. The catheter of claim 4 wherein a tag is attached to the distal end of the agent.

9. The catheter of claim 8 wherein the tag is magnetic.

10. The catheter of claim 8 wherein the tag is non-magnetic.

11. The catheter of claim 10 wherein the tag is a radio frequency identification tag.

12. The catheter of claim 1 wherein a tag is embedded in the body at or near the distal end of the body.

13. The catheter of claim 1 wherein a tip is contiguous to the distal end of the body.

14. The catheter of claim 13 wherein a tag is embedded in the tip.

15. The catheter of claim 12 or 14 wherein the tag is magnetic.

16. The catheter of claim 12 or 14 wherein the tag is non-magnetic.

17. The catheter of claim 16 wherein the tag is a radio frequency identification tag.

18. A catheter for use in the delivery and/or the withdrawal of one or more substances to a patient, wherein the catheter comprises a body having a proximal end and a distal end wherein the body encloses at least one independent lumen which extends longitudinally from the proximal end to the distal end, wherein a tip is contiguous to the distal end of the body.

19. The catheter of claim 18 wherein the proximal end of the body is attached to a totally implantable venous access system.

20. The catheter of claim 18 wherein a tag is embedded in the tip.

21. The catheter of claim 18 wherein a tag is embedded in the body at or near the distal end of the body.

22. The catheter of claim 20 or 21 wherein the tag is magnetic.

23. The catheter of claim 20 or 21 wherein the tag is non-magnetic.

24. The catheter of claim 23 wherein the tag is a radio frequency identification tag.

25. The catheter of claim 20 or 21 wherein the proximal end of the body is attached to a totally implantable venous access system.

26. The catheter of claim 25 wherein the tag is magnetic.

27. The catheter of claim 25 wherein the tag is non-magnetic.

28. The catheter of claim 27 wherein the tag is a radio frequency identification tag.

29. A method for stiffening a catheter comprising:
a.) obtaining a catheter for use in the delivery and/or the withdrawal of a variety of substances to a patient, wherein the catheter comprises a body having a proximal end and a distal end wherein the body encloses a plurality of independent lumens which extend longitudinally from the proximal end to the distal end, wherein at least one of the lumens is sealed at the distal end which allows for the insertion of a agent;
b.) inserting the agent into the at least one of the lumens that is sealed at the distal end;
c.) inserting the catheter into a patient;
d.) securing the catheter to the patient;
e.) delivering and/or withdrawing a substance to and/or from the patient; and
f.) removing the agent.

30. The method of claim 29 wherein the agent is a solid, liquid or gas.

31. The method of claim 29 wherein the method further comprises the steps:
g.) reinserting the agent into the at least one of the lumens that is sealed at the distal end;
h.) delivering and/or withdrawing a substance to and/or from the patient; and
i.) removing the agent.

32. A method for temporarily stiffening a catheter for use in the delivery and/or the withdrawal of a variety of substances to a patient, wherein the catheter comprises a body having a proximal end and a distal end wherein the body encloses a plurality of independent lumens which extend longitudinally from the proximal end to the distal end, wherein at least one of the lumens is sealed at the distal end which allows for the insertion of a agent, wherein the catheter has previously been inserted and secured to a patient, the method comprising the steps:
a.) inserting the agent into the at least one of the lumens that is sealed at the distal end;
b.) delivering and/or withdrawing a substance to and/or from a patient; and
c.) removing the agent.

33. The method of claim 31 or 32 wherein the agent is a solid, liquid or gas.

34. A method for identifying the position of and/or communicating with the distal end of a catheter comprising:
a.) obtaining a catheter for use in the delivery and/or the withdrawal of one or more substances to a patient, wherein the catheter has a body having a proximal end and a distal end wherein the body encloses at least one lumen which extends longitudinally from the proximal end to the distal end, wherein a tag is located near or at the distal end of the body;
b.) inserting the catheter into a patient;
c.) securing the catheter to the patient; and
d.) using identification technology to identify the position of and/or communicate with the tag.

35. The method of claim 34 wherein the tag is embedded in a tip, wherein the tip is contiguous to the distal end of the body.

36. A method for identifying the position of and/or communicating with a tag located on an agent comprising:
a.) obtaining a catheter for use in the delivery and/or the withdrawal of a variety of substances to a patient, wherein the catheter has a body having a proximal end and a distal end wherein the body encloses a plurality of lumens which extend longitudinally from the proximal end to the distal end, wherein at least one of the lumens is sealed at the distal end which allows for the insertion of an agent, wherein the agent has a proximal end and a distal end, wherein a tag is attached to the distal end of the agent;
b.) inserting the agent into one of the lumens which is sealed at the distal end;
c.) inserting the catheter into a patient;
d.) securing the catheter to the patient;
e.) using identification technology to identify the position of and/or communicate with the tag; and
f.) removing the agent.

37. The method of claim 36 further comprising the steps:
g.) reinserting the agent into one of the lumens which is sealed at the distal end; and
h.) using identification technology to identify the position of and/or communicate with the tag.

38. The method of claim 36 or 37 wherein the agent is a solid.

39. A method for identifying the position of and/or communicating a tag located on an agent used in conjunction with a catheter, wherein the catheter is for use in the delivery and/or the withdrawal of a variety of substances to a patient, wherein the catheter has a body having a proximal end and a distal end wherein the body encloses a plurality of lumens which extend longitudinally from the proximal end to the distal end, wherein at least one of the lumens is sealed at the distal end which allows for the insertion of an agent, wherein the agent has a distal end, wherein a tag is attached to the distal end of the agent, wherein the catheter has been inserted into the patient, the method comprising the steps:
a.) inserting the agent into one of the lumens which is sealed at the distal end until the distal end of the agent is at or near the distal end of the catheter and
b.) using identification technology to identify the position of and/or communicate with the tag.

40. The method of claim 39 wherein the agent is a solid.

41. A method for preventing the distal end of a catheter from irritating or injuring any body duct, cavity or tissue, and/or causing open lumen occlusion, the method comprising:
a.) obtaining a catheter for the delivery and/or withdrawal of one or more substances to a patient, wherein the catheter comprises a body having a proximal end and a distal end wherein the body encloses at least one independent lumen which extends longitudinally from the proximal end to the distal end, wherein a tip is contiguous to the distal end of the body;
b.) inserting the catheter into a patient;
c.) securing the catheter to the patient; and
d.) delivering and/or withdrawing a substance to and/or from the patient.
